(19) United States
(12) Patent Application Publication
(10) Pub. No.: US 2008/0221021 A1
(43) Pub. Date: Sep. 11, 2008

(54) Novel Corticotropin-Releasing Factor Receptor 1 (Crfr1) Agonist

(75) Inventors: Joachim Spiess, Gottingen (DE); Olaf Jahn, Gottingen (DE); Hossein Tezval, Gottingen (DE)

Correspondence Address: BIRCH STEWART KOLASCH & BIRCH, PO BOX 747, FALLS CHURCH, VA, 22040-0747, US

(73) Assignee: Max-Planck-Gesellschaft Zur Forderung Der, Berlin (DE)

(21) Appl. No.: 10/586,469

(22) PCT Filed: Jan. 14, 2005
(86) PCT No.: PCT/EP05/00356

§ 371 Date: Dec. 18, 2006

(30) Foreign Application Priority Data
Jan. 19, 2004 (EP) 04001009.2

(51) Int. Cl. A61K 38/16; C07K 14/00; C07H 21/04; C12N 15/64; A61K 31/7088; A61P 25/24; A61P 5/06; A61P 25/00

(52) U.S. Cl. 514/12; 530/324; 536/23.1; 514/44; 435/320.1

(57) ABSTRACT

The present invention relates to a compound which is highly selective for CRFR1 without having any significant cross-reactivity for corticotropin-releasing-factor-receptor-2 (CRFR2) and/or corticotropin-releasing-factor-binding protein (CRFBP), said compound comprising or alternatively consisting of the amino acid sequence as depicted in SEQ ID No: 1. In another aspect, the present invention relates to a pharmaceutical and/or diagnostic composition comprising the novel CRFR1 agonist of the present invention. The present invention also provides a kit comprising the novel CRFR1 agonist of the present invention and optionally instructions to use. Furthermore, the present invention provides the use of the compound of the present invention for the preparation of a pharmaceutical composition for the treatment of depression and, additionally, the use of the compound of the present invention for the preparation of a diagnostic composition for the determination of pituitary corticotroph responsiveness and/or for differentiating pituitary and ectopic production of ACTH in patients with ACTH-dependent Cushing's syndrome.

The present invention relates to a compound which is highly selective for CRFR1 without having any significant cross-reactivity for corticotropin-releasing-factor-receptor-2 (CRFR2) and/or corticotropin-releasing-factor-binding protein (CRFBP), said compound comprising or alternatively consisting of the amino acid sequence as depicted in SEQ ID No: 1. In another aspect, the present invention relates to a pharmaceutical and/or diagnostic composition comprising the novel CRFR1 agonist of the present invention. The present invention also provides a kit comprising the novel CRFR1 agonist of the present invention and optionally instructions to use. Furthermore, the present invention provides the use of the compound of the present invention for the preparation of a pharmaceutical composition for the treatment of depression and, additionally, the use of the compound of the present invention for the preparation of a diagnostic composition for the determination of pituitary corticotroph responsiveness and/or for differentiating pituitary and ectopic production of ACTH in patients with ACTH-dependent Cushing's syndrome.

A variety of documents is cited throughout this specification. The disclosure content of said documents (including any manufacturer's specifications, instructions etc.) is herewith incorporated by reference; however, there is no admission that any document cited is indeed prior art as to the present invention.

Corticotropin-releasing factor (CRF), a C-terminally amidated neuropeptide of 41 amino acids (1), is the major regulator of the hypothalamus-pituitary-adrenal (HPA) axis (2). In addition, CRF modulates a variety of important brain functions such as anxiety, learning, food intake, and locomotion (3, 4) and is linked to the pathogenesis of anxiety disorders and depression. CRF acts through two known G protein dependent CRF receptor (CRFR) subtypes, CRFR1 and CRFR2, derived from two different genes (5). In addition, CRF binds with high affinity to a binding protein (CRFBP) proposed to function as a pharmacologically significant reservoir of endogenous CRF (6, 7). Several CRFR1 and CRFR2 splice variants have been described (5, 8). However, the majority of these variants are restricted to humans (8). In rodents, it appears that only the splice variants CRFR1α, CRFR2α, and CRFR2β are of physiological relevance (5). In rodents, CRFR2α mRNA is primarily expressed in the central nervous system, whereas CRFR2β mRNA is found in non-neuronal brain structures such as the choroid plexus cerebral arterioles and in the periphery (8).

The two known CRFR subtypes are differently involved in biological functions (3). Thus, activation of the hypothalamic-pituitary-adrenal (HPA)-axis in response to a stressful stimulus is mainly achieved through CRFR1 (10). In contrast, both CRFR subtypes modulate anxiety-like behavior in a complex manner. It was demonstrated by gene deletion experiments that anxiety-like behavior is predominantly enhanced through CRFR1 (11, 12), whereas it is reduced through CRFR2 (10, 13). Pharmacological experiments discriminating between regional actions of CRFR1 and CRFR2 revealed that CRFR2 of the lateral intermediate septum mediates stress-induced enhancement of anxiety-like behavior (23), whereas CRFR2 accessed through the brain ventricles is anxiolytic (13). In contrast, CRFR1 accessed by CRF via the brain ventricles enhances anxiety-like behavior (21). Several natural CRF-like peptides with different CRFR subtype specificity have been characterized. Thus, human/rat CRF (h/rCRF) and ovine CRF (oCRF) exhibit a partial preference for CRFR1, whereas urocortin I (UcnI) (15) is a non-selective ligand (3, 16). Recently, urocortin II (Ucn II) (17) also described as stresscopin-related peptide (SRP) (18) and urocortin III (Ucn III) (19), also called stresscopin (SCP) (18) were identified on the basis of homology analysis of data derived from genomic sequence databases. With the finding of Ucn II and Ucn III, highly selective agonists for CRFR2 have been discovered. In contrast, neither natural nor synthetic agonists with a similar selectivity for CRFR1 have been identified to date.

At this time, oCRF is the agonist of choice for the selective stimulation of CRFR1 in behavioral experiments, because it displays a preference for CRFR1 over CRFR2 of two orders of magnitude as determined by ligand binding assays (16, 21). However, due to the high local agonist concentration that often occurs when drugs are directly administered into the animal brain, the use of oCRF for the stimulation of CRFR1 may lead to CRFR2-mediated side effects (Todorovic and Spiess, unpublished data). For example, injection of 100 ng OCRF into the septum, a brain region containing predominantly CRFR2, induces a significant CRFR2-mediated anxiogenic behavior in the mouse. In addition, displacement of endogenous ligand from CRFBP (6) by the agonist applied may release endogenous CRF-like peptide and thus interfere with the desired selective stimulation of CRFR1.

In summary, the two different subtypes of the CRFR differently affect brain functions such as anxiety and memory under physiological and pathophysiological conditions. In view of the complex involvement of CRFR1 in brain function, the availability of a selective and highly potent agonist for this receptor subtype is of great interest.

The solution to this technical problem is achieved by providing the embodiments characterized in the claims.

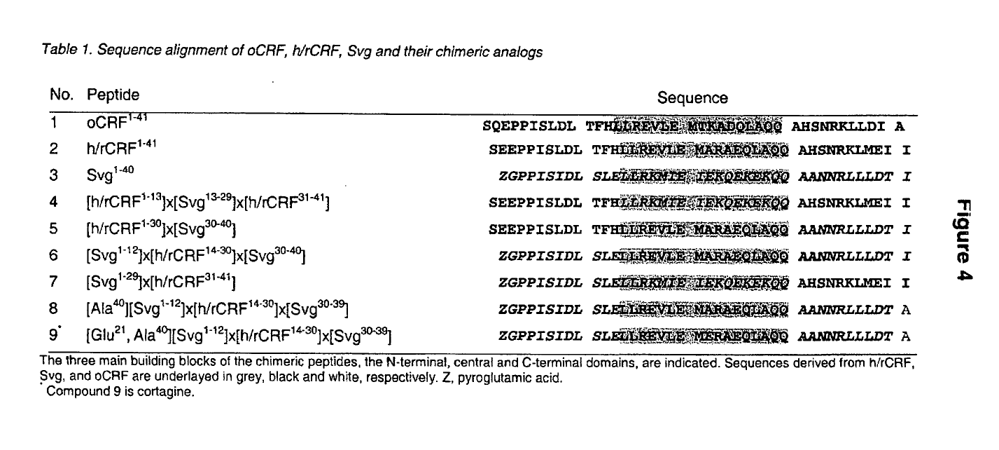

Chimeric peptides were designed on the basis of the amino acid sequences of the rodent peptide h/rCRF and oCRF preferentially binding to CRFR1 and the non-selective frog peptide Svg (FIG. 4) because of its advantageous physicochemical properties, Due to its relatively high hydrophilicity and relatively low isoelectric point (21), Svg is anticipated to be soluble under physiological conditions. Therefore, Svg was selected for the chimeric peptide approach, because a high solubility under physiological conditions is a crucial prerequisite for a CRFR1 agonist to be applied in behavioral experiments.

On the basis of the recent finding that CRF contains segregated receptor binding sites at its N- and C-termini (25), the sequence of CRF was divided into an N-terminal (residues 1-13), a central (residues 14-30), and a C-terminal (residues 31-40) domain (FIG. 4), to be used as building blocks for the initial design of the chimeric peptides. These domains derived from the sequence of h/rCRF and Svg, respectively, were mixed to generate the chimeric peptides [h/rCRF¹⁻¹³]x[Svg¹³⁻²⁹]x[h/rCRF³¹⁻⁴¹] (FIG. 4, compound 4), [h/rCRF¹⁻³⁰]x[Svg³⁰⁻⁴⁰] (compound 5), [Svg¹⁻¹² ]x[h/rCRF¹⁴⁻³⁰]x[Svg³⁰⁻⁴⁰] (compound 6), and [Svg¹⁻²⁹]x[h/rCRF³¹⁻⁴¹] (compound 7). An exchange of the N-terminal parts of h/rCRF and Svg was not considered because it is unlikely that these parts contain a motif controlling the affinity to CRFR2 as can be concluded from the observation that binding to CRFR2 is not sensitive to the truncation of h/rCRF (26) or Svg (20).

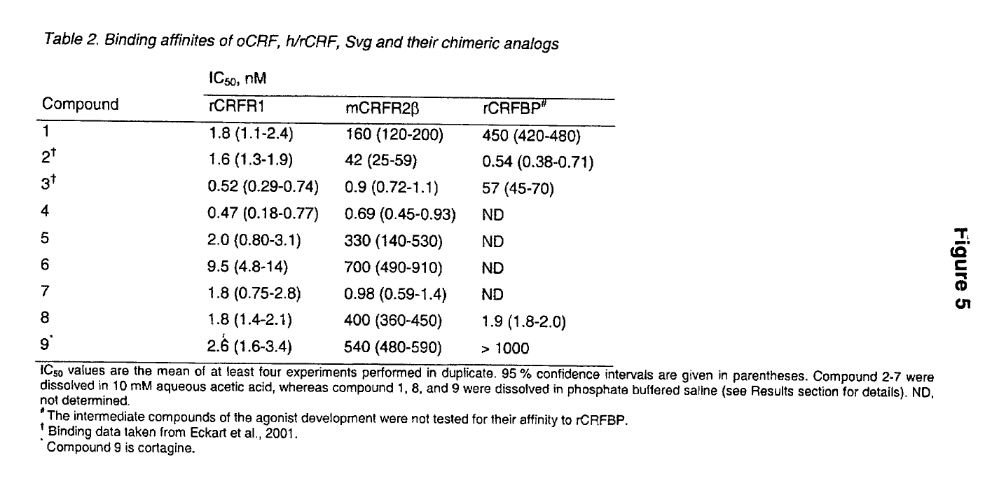

As determined by binding analysis, compound 5, but not compound 4, exhibited a low affinity for CRFR2 and a clear selectivity for CRFR1 (FIG. 5). Therefore, it was concluded that residues 14-30 of h/rCRF contain the motif responsible for a decrease in affinity to CRFR2. In agreement with this conclusion, compound 6, but not compound 7, was selective for CRFR1 (FIG. 5). On the basis of its low affinity to CRFR2, compound 6 was selected for the optimization of its pharmacological properties to develop a CRFR1 selective agonist. An additional rationale for the selection of compound 6 as lead compound was that it contains an N-terminal pyroglutamic acid derived from the Svg sequence (FIG. 4). The presence of this cyclic residue may prevent degradation by major aminopeptidases that require a free α-amino group for their action (27). It was therefore anticipated that the blocked N-terminus provides compound 6 or analogs thereof with an increased stability in in vivo experiments.

Further amino acid replacements of compound 6 were considered (i) to improve its selectivity for CRFR1 and (ii) to prevent binding to CRFBP. Although h/rCRF and oCRF differ by only seven amino acids (position 2, 22, 23 25, 38, 39, and 41; FIG. 4), oCRF exhibited an approximately three-fold higher selectivity for CRFR1 than h/rCRF as determined by binding analysis (FIG. 5). On the basis of the observation that neither the N-terminal residue Glu² nor the central residues Ala²², Arg²³, and Glu²⁵ of h/rCRF have a significant influence on receptor selectivity (21, 26), only the C-terminal residues in positions 38, 39, and 41 were considered for amino acid replacements. This comparison of the sequences of oCRF, h/rCRF, Svg, and compound 6 revealed that the CRFR1-selective oCRF and the non-selective Svg share residues Leu³⁸ and Asp³⁹ (FIG. 4). It was therefore hypothesized that Ala⁴¹ of oCRF significantly contributes to the binding preference of this ligand. This hypothesis was first tested by the synthesis and characterization of [Ala⁴¹]h/rCRF. In comparison to the parent peptide h/rCRF, [Ala⁴¹]h/rCRF showed an increase in CRFR1 selectivity by a factor of three, and, thus a similar selectivity as oCRF (data not shown). As expected on the basis of this result, a peptide highly selective for CRFR1 was obtained when the same replacement was carried out for compound 6 to generate [Ala⁴⁰] [Svg¹⁻¹²]x[h/rCRF¹⁴⁻³⁰]x[Svg³⁰⁻⁴⁰] (compound 8). In comparison to compound 6, compound 8 showed a more than five-fold increase in affinity to CRFR1 that was accompanied by only a slight increase in affinity for CRFR2 (FIG. 5).

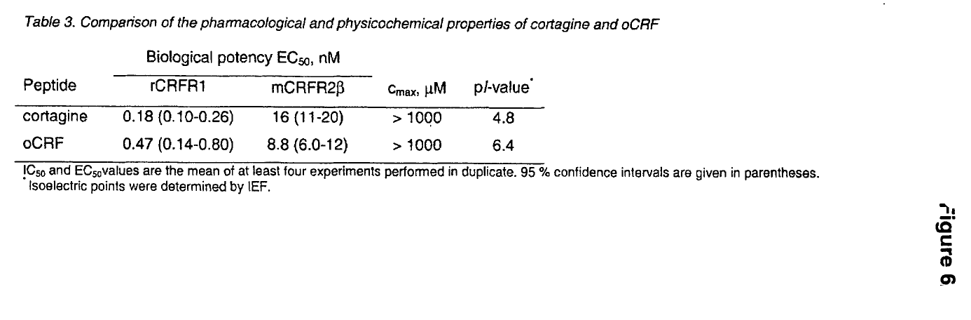

A high solubility under physiological conditions is a crucial prerequisite especially for compounds to be injected into the rodent brain. Therefore, the maximal solubility of cortagine was determined. It was observed that cortagine like oCRF were soluble in a concentration range of up to 1000 μM (FIG. 6), so that there was no limitation for behavioral experiments in view of the agonist doses typically used.

The next step in the agonist development was to remove the high affinity of compound 8 to CRFBP (FIG. 5) by employing the recently reported single amino acid switch concept determining the affinity to CRFBP (21). Therefore, Ala²¹ of compound 8 was replaced by a Glu residue, an exchange that has been shown to decrease the affinity of h/rCRF to CRFBP by two orders of magnitude (21). Thus, [Glu²¹, Ala⁴⁰] [Svg¹⁻¹²]x[h/rCRF¹⁴⁻³⁰]x[Svg³⁰⁻⁴⁰ ] (compound 9) was obtained and tested for its affinity to CRFR1, CRFR2, and CRFBP. As expected, no significant change in receptor binding was observed, whereas the affinity to CRFBP was completely abolished (FIG. 5). Met²⁰ of compound 9 was substituted by a norleucine residue to prevent the formation of methionine sulfoxide, a modification that is known to abolish the bioactivity of CRF-like peptides (2). However, this amino acid replacement led to a significant decrease in affinity for CRFR1 (data not shown). Thus, compound 9 was the final compound of our development strategy and selected for further pharmacological, physicochemical, and behavioral characterization in comparison with the reference compound oCRF. Because compound 9 is mainly composed of domains derived from the sequences of h/rCRF and Svg, respectively, it was named cortagine.

By using a chimeric peptide approach, a new potent agonist selective for CRFR1, [Glu²¹, Ala⁴⁰] [Svg¹⁻¹²]x[h/rCRF¹⁴⁻³⁰]x[Svg³⁰⁻⁴⁰] (compound 9), has been developed and named cortagine. The design strategy that finally led to the development of cortagine was to identify amino acid residues of h/rCRF and oCRF which affect receptor selectivity. On the basis of the pharmacological properties of the different chimeric peptides, a selective decrease in affinity to CRFR2 was attributed to the central part of h/rCRF (residues 14-30), while the presence of the C-terminal Ala residue was shown to correlate with a selective increase in affinity to CRFR1. Therefore, these residues were introduced into the sequence of the non-selective peptide Svg and combined with an Ala-Glu exchange in position 21 known to abolish high affinity to CRFBP (21). The resulting peptide, cortagine, displayed a high agonistic potency at CRFR1, whereas the cross-reactivities to CRFR2 and CRFBP were negligible.

The pharmacological characterization of cortagine revealed a selectivity for CRFR1 higher than that of the reference compound oCRF. As calculated on the basis of the binding affinities (IC₅₀(mCRFR2)/IC₅₀(rCRFR1); FIG. 5), a CRFR1-selectivity of 208 over CRFR2β was found for cortagine, whereas oCRF displayed a selectivity of only 89. The improved selectivity of cortagine was also reflected by ratios calculated on the basis of the biological potencies (EC₅₀(mCRFR2)/EC₅₀(rCRFR1); FIG. 6). A CRFR1-selectivity of 89 over CRFR2β, was found for cortagine, whereas oCRF displayed a selectivity of only 19.

Intraventricular application of cortagine successfully induced enhancement of anxiety-like behavior and decreased locomotor activity of the mouse in the EPM at doses lower than required of OCRF. The behavioral effects of cortagine were mediated by CRFR1, as indicated by the observation that activation of CRFR1 in a novel environment results in reduction of locomotor activity (30). Importantly, intraseptal application of oCRF, but not of cortagine, produced an anxiogenic effect which was completely prevented by aSvg-30, suggesting that septal CRFR2 mediated the anxiogenic effects of oCRF. Thus, our experiments in vivo demonstrated that, in contrast to oCRF, cortagine lacks any significant cross reactivity to CRFR2.

The unequivocal behavioral effects of cortagine may be due to a combination of at least three beneficial properties. First, as already discussed above, cortagine exhibits a high selectivity for rCRFR1 over mCRFR2β. We are aware of the possible draw back that recombinant mCRFR2β was employed for the development of cortagine, whereas the target of the behavioral experiments was CRFR2α, the predominant splice variant of CRFR2 in the rodent brain (9). However, on the basis of reported observations that mouse CRFR2β and rat CRFR2α do not differ significantly in their pharmacological profile (5, 19), it is hypothesized that cortagine's low affinity and biological potency also holds true for mouse CRFR2α. This hypothesis is not only supported by the results of the behavioral experiments presented here, but also by preliminary binding data using HEK 293 cells stably expressing cloned mouse CRFR2α, a new cell line that is currently established in our laboratory (Zeyda and Spiess, unpublished data). Second, cortagine does not display any detectable affinity for CRFBP proposed to function as a reservoir of endogenous CRF (6). Thus, it can be excluded that the effective dose of cortagine is decreased due to capture of the ligand by CRFBP. Furthermore, displacement of endogenously bound ligand from CRFBP, an effect that may interfere with the desired selective stimulation of CRFR1 by dilution of cortagine with released ligand, can be also ruled out. Third, it is likely that the blocked N-terminus of cortagine provides the peptide with an improved stability in vivo which may be accompanied with a longer duration of action.

By the development of cortagine as a new CRFR1-selective agonist and the recent discovery of the endogenous, CRFR2-selective ligands (17-19), useful tools became available for the analysis of unique or overlapping roles of CRFR subtypes in complex physiological functions such as anxiety and HPA axis regulation. Especially in brain regions where the CRFR subtypes are co-localized, e.g. medial and basolateral nuclei of amygdala, bed nucleus of stria terminal, hippcampal formation, and raphe nuclei (31), the use of cortagine will substantially facilitate the investigation of CRFR1-mediated effects.

Accordingly, the present invention relates to a compound which is highly selective for CRFR1 without having any significant cross-reactivity for corticotropin-releasing-factor-receptor-2 (CRFR2) and/or corticotropin-releasing-factor-binding protein (CRFBP), said compound comprising or alternatively consisting of the amino acid sequence Glx¹-Gly²-Pro³-Pro^{4-Xaa}⁵ -Ser⁶ -Xaa⁷-Asp⁸-Leu⁹-Xaa¹⁰-Leu¹¹-Glu¹²-Leu¹³-Leu¹⁴-Arg¹⁵-Glu¹⁶-Val¹⁷-Leu¹⁸-Glu¹⁹-Xaa²⁰-Xaa²¹-Arg²²-Ala²³-Xaa²⁴-Gln²⁵-Leu²⁶-Ala²⁷-Gln²⁸-Gln²⁹-Ala³⁰-Ala³¹-Asn³²-Asn³³-Arg³⁴-Leu³⁵-Leu³⁶-Leu³⁷-Asp³⁸-Thr³⁹-Ala⁴⁰ (SEQ ID No: 1).

It must be noted that as used herein, the singular forms “a”, “an”, and “the”, include plural references unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

The term “highly selective for CRFR1 without having any significant cross-reactivity for corticotropin-releasing-factor-receptor-2 (CRFR2) and/or corticotropin-releasing-factor-binding protein (CRFBP)” as used herein means that the IC₅₀ values for rCRFR1, mCRFR2β and rCRFBP of the compounds of the present invention deviate from the IC₅₀ values for rCRFR1, mCRFR2β and rCRFBP of compound 9 which is depicted in FIG. 6 (or SEQ ID No:2), not more than 15%, preferably not more than 10%, 7.5%, 5% and even more preferably not more than 2.5%, 2%, 1.5%, 1%, 0.5% or 0.25%. Methods for measuring the mentioned IC₅₀ values for rCRFR1, mCRFR2β and rCRFBP are well-known to the skilled person and furthermore explained in detail in the appended examples.

Thus, in a preferred embodiment, the compound of the present invention as mentioned herein before is characterized by one or more of the following amino acid exchanges which are characterized as follows:
(a) Xaa⁵ is Ile, Leu or an amino acid residue having similar physicochemical characteristics; and/or
(b) Xaa⁷ is Ile, Leu or an amino acid residue having similar physicochemical characteristics; and/or
(c) Xaa¹⁰ is Ser, Thr or an amino acid residue having similar physicochemical characteristics; and/or
(d) Xaa²⁰ is Met, Norleucine or any amino acid residue having similar physicochemical characteristics; and/or
(e) Xaa²¹ is Glu, Asp or an amino acid residue having similar physicochemical characteristics; and/or
(f) Xaa²⁴ is Glu, Asp or an amino acid residue having similar physicochemical characteristics.

The term “amino acid residue(s)” as mentioned herein encompasses amino acid derivatives like chemically modified amino acid residues which do not alter or do not essentially alter the “physicochemical properties” of the above specified amino acids as depicted in sections (a) to (f). The term “derivatives” or “chemical derivatives” is further explained herein below.

In a most preferred embodiment of the compound of the present invention, said compound (which is named cortagine) comprises or alternatively consists of the amino acid sequence Glx¹-Gly²-Pro³-Pro⁴-Ile⁵-Ser⁶-Ile⁷-Asp⁸-Leu⁹-Ser¹⁰-Leu¹¹-Glu¹²-Leu¹³-Leu¹⁴-Arg¹⁵-Glu¹⁶-Val¹⁷-Leu¹⁸-Glu¹⁹-Met²⁰-Glu²¹-Arg²²-Ala²³-Glu²⁴-Gln²⁵-Leu²⁶-Ala²⁷-Gln²⁸-Gln²⁹-Ala³⁰-Ala³¹-Asn³²-Asn³³-Arg³⁴-Leu³⁵-Leu³⁶-Leu³⁷-Asp³⁸-Thr³⁹-Ala⁴⁰ (depicted in SEQ ID No: 2). The latter sequence is also named compound 9 which is depicted in FIG. 6 herein below.

In a further embodiment the present invention relates to a nucleic acid molecule encoding the compound of the present invention, preferably the compound as depicted in SEQ ID No: 1 or 2. In addition, also such nucleic acid molecules are within the scope of the present invention which hybridize under stringent conditions with a nucleic acid molecule encoding SEQ ID No: 1 or 2 (or compound 9 as depicted in FIG. 6 herein below) and encode a compound which is highly selective for CRFR1 without having any significant cross-reactivity for corticotropin-releasing-factor-receptor-2 (CRFR2) and/or corticotropin-releasing-factor-binding protein (CRFBP). The meaning of the term “compound which is highly selective for CRFR1 without having any significant cross-reactivity for corticotropin-releasing-factor-receptor-2 (CRFR2) and/or corticotropin-releasing-factor-binding protein (CRFBP)” has been described elsewhere herein. Preferably, the compound of the present invention is a peptide, protein or polypeptide (i.e. a peptide-compound, protein compound or polypeptide-compound).

The term “stringent conditions” as used herein refers to an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. Such methods are well-known in the art.

The nucleic acid molecule of the present invention may be, e.g., DNA, cDNA, RNA or synthetically produced DNA or RNA or a recombinantly produced chimeric nucleic acid molecule comprising any of those nucleic acid molecules either alone or in combination.

In another embodiment the present invention relates to a vector comprising the nucleic acid molecule of the present invention. The vector of the present invention may be, e.g., a plasmid, cosmid, virus, bacteriophage or another vector used conventionally in genetic engineering, and may comprise further genes such as marker genes which allow for the selection of said vector in a suitable host cell and under suitable conditions. In a preferred embodiment of the vector of the present invention the nucleic acid molecule is operatively linked to an expression control sequence. Said expression control sequence allows expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory elements permitting expression in prokaryotic host cells comprise, e.g., the PL, lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (Invitrogene), or pSPORT1 (GIBCO BRL). Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences.

Furthermore, the present invention relates to a non-human host comprising the nucleic acid molecule or vector of the present invention. Said host may be a prokaryotic or eukaryotic cell. The nucleic acid molecule or vector of the invention which is present in the host cell may either be integrated into the genome of the host cell or it may be maintained extrachromosomally.

The host cell can be any prokaryotic or eukaryotic cell, such as a bacterial, insect, fungal, plant, animal or human cell. Preferred fungal cells are, for example, those of the genus Saccharomyces, in particular those of the species S. cerevisiae. The term “prokaryotic” is meant to include all bacteria which can be transformed or transfected with a polynucleotide or vector of the present invention for the expression of the antagonist of the present invention. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells. Depending upon the host employed in a recombinant production procedure, the compound encoded by the nucleic acid molecule and/or vector of the present invention may or may not be post-translationally modified. A nucleic acid molecule of the invention can be used to transform or transfect the host using any of the techniques commonly known to those of ordinary skill in the art. Furthermore, methods for preparing fused, operably linked genes and expressing them in, e.g., mammalian cells and bacteria are well-known in the art (e.g. Sambrook et al., Molecular Cloning: A Laboratory Manual, Cold Spring Harbor Laboratory, Cold Spring Harbor, N.Y., 1989). The genetic constructs and methods described therein can be utilized for expression of the compound of the present invention in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Furthermore, transgenic animals, preferably mammals, comprising host cells of the invention may be used for the large scale production of the compound of the present invention.

Alternatively, it is of course possible to chemically synthesize the compound of the present invention , e.g. by means and methods well known in the art, e.g., solid phase synthesis with Fmoc or t-boc chemistry (see also , e.g., Rühmann, A., A. K. E. Köpke, F. M. Dautzenberg, and J. Spiess, Proc. Natl. Acad. Sci. USA 93:10609-10613, 1996).

In another embodiment, the compound of the present invention is labelled with an appropriate marker or tag for specific applications, such as for the detection of the presence of the CRFR1-receptor in a sample, e.g. a sample derived from an organism, in particular mammals, preferably human. A number of companies such as Promega (Madison Wis.), and US Biochemical Corp (Cleveland Ohio) supply commercial kits and protocols for these procedures. Suitable reporter molecules or labels include radionuclides such as but not limited to iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin, enzymes (like horse radish peroxidase, β-galactosidase, alkaline phosphatase), chemi- or bioluminescent compounds (like dioxetanes, luminol or acridiniums), fluorochromes (like fluorescein, rhodamine, Texas Red, etc.) or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat No. 3,939,350; U.S. Pat No. 3,996,345; U.S. Pat. No. 4,227,437; U.S. Pat. No. 4,275,149 and U.S. Pat. No. 4,366,241. It is also envisaged that said tag is selected, but not limited to, from the group consisting of His-tag, Streptavidin-tag, HA-tag, GST-tag, CBP-tag, MBP-tag, FLAG-tag, myc as well as single-chain fragments (sc Fvs) of antibody binding regions. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, RH and von Knippenburg (Eds), Volume 15 (1985), “Basic methods in molecular biology”; Davis LG, Dibmer MD; Battey Elsevier (1990), Mayer et al., (Eds) “Immunochemical methods in cell and molecular biology” Academic Press, London (1987), or in the series “Methods in Enzymology”, Academic Press, Inc. There are many different labels and methods of labeling known to those of ordinary skill in the art.

Labeling procedures, like covalent coupling of enzymes or biotinyl groups, iodinations, phosphorylations, biotinylations, are well known in the art. Detection methods comprise, but are not limited to, autoradiography, fluorescence microscopy, direct and indirect enzymatic reactions, FACS-analysis etc.

It is also envisaged that the compounds of the present invention are labelled with a photoactivatable group such as a benzophenone moiety provided by para-benzoylbenzoic acid or para-hydroxybenzoylbenzoic acid. Means and methods for achieving such a labeling with a photoactivatable group are explained in great detail in WO 02/095395, which is therefore incorporated herein by reference. A further label which is also within the scope of the present invention is a gadolinium-label.

The person skilled in the art is well aware that such labels as mentioned herein before should have no or essentially no, i.e. only a minor effect on the binding characteristics of the compound of the present invention. The skilled person is aware of methods of how to measure such binding characteristics which are known in the art and which are also disclosed in great detail in the appended examples and, thus, guide the skilled person when designing such a test system. It is envisaged that coupling a label and/or a tag as described herein changes the binding characteristics of said labeled compound to CRFR1, CRFR2 and CRFBP (i.e. the labeled or otherwise modified compounds are still highly selective for CRFR1 without having any significant cross-reactivity for corticotropin-releasing-factor-receptor-2 (CRFR2) and/or corticotropin-releasing-factor-binding protein (CRFBP as explained herein) not more than 40%, 30%, 25%, 20%, 15%, 10%, 7.5%, 5%, 2.5%, 2%, 1%, 0.5% and/or not more than 0.1% when compared with the respective non-labeled non-modified or non-tagged compound.

It is also envisaged that the compound of the present invention is protected against peptidases by means and methods known in the art e.g. by incorporation of a D-amino acid residues or by acetylation e.g of the N-terminus of the compound of the invention.

The present invention also relates to chemical derivatives of the compound of the invention. The “chemical derivative” contains additional chemical moieties which are not normally a part of the peptide-compound (as depicted for example in SEQ ID No: 1 or 2) and is encompassed by the invention as long as it retains at least a portion of the function of the compound which are highly selective for CRFR1 without having any significant cross-reactivity for corticotropin-releasing-factor-receptor-2 (CRFR2) and/or corticotropin-releasing-factor-binding protein (CRFBP). For example, a chemical derivative may result from the reaction of an organic derivatizing agent capable of reacting with selected side chains or terminal residues of the compound of the invention, and will preferably retain at least a portion of the function of the compound of the invention, i.e. it is highly selective for CRFR1 without having any significant cross-reactivity for corticotropin-releasing-factor-receptor-2 (CRFR2) and/or corticotropin-releasing-factor-binding protein (CRFBP). Among these chemical derivatives, the amides are of particular interest, both amides of carboxyl groups at the C-terminus and amides of free carboxyl groups of aspartic or glutamic acid residues. Many such chemical derivatives and methods for making them are well known in the art.

In a further embodiment, the present invention relates to the compound as described herein which is modified by:
(a) formation of pharmaceutically acceptable salts;
(b) formation of pharmaceutically acceptable complexes; and/or
(c) synthesis of pharmacologically active polymers.

As used herein, the term “pharmaceutically acceptable salts” refers to both salts of carboxyl groups and to acid addition salts of amino groups of the compound of the invention. Salts of a carboxyl group may be formed by means and methods known in the art and include inorganic salts, for example, sodium, calcium, ammonium, ferric or zinc salts, and the like, and salts with organic bases such as those formed for example, with amines, such as triethanolamine, arginine, or lysine, piperidine, procaine, and the like. Acid addition salts include, for example, salts with mineral acids such as, for example, hydrochloric acid or sulfuric acid, and salts with organic acids, such as, for example, acetic acid or oxalic acid. The above mentioned chemical derivatives of the compound of the invention as well as the salts as described herein are preferably used to modify the pharmaceutical properties of the compound insofar as stability, solubility, etc., are concerned.

It is also envisaged that the compound of the present invention is a multiepitope single peptide comprising a number of the same or different compounds of the present invention in the form of a peptide polymer (i.e. in the form of a “pharmacologically active polymers” as mentioned herein), obtained, for example, by polymerization of the compounds of the invention with a suitable polymerization agent, such as 0.1% glutaraldehyde (Audibert et al., 1981, Nature 289:593). The polymer will preferably contain 2, 3, 4, 5, 6, 7, 8, 9, 10, 20, 30, 40, or 50 units of the compound of the invention, preferably a peptide-compound as depicted in SEQ ID NO: 1 or 2. Such peptide polymers may also be formed by crosslinking the compounds or attaching multiple peptide-compounds to macromolecular carriers which results in the pharmaceutically acceptable complexes as mentioned herein. Suitable macromolecular carriers are, for example, proteins, such as tetanus toxoid, and linear or branched copolymers of amino acids, such as a linear copolymer of L-alanine, L-glutamic acid and L-lysine and a branched copolymer of L-tyrosine, L-glutamic acid, Lalanine and L-lysine (T, G)-A-L-, or multichain poly-DL-alanine (M. Sela et al., 1955, J. Am. Chem. Soc. 77: 6175). The conjugates are obtained, for example, by first coupling the peptide with a water-soluble carbodiimide, such as I-ethyl-3-(3′-dimethylamino-propyl) carbodiimide hydrochloride, and then performing the conjugation with the macromolecular carrier.

In behavior experiments on mice for the modulatory action of the compounds of the invention on anxiety- and depression-like behaviors using the elevated plus-maze (EPM) test and the forced swim test (FST), respectively, cortagine reduced significantly the floating time in the FST as described for anti-depressive drugs.

Thus, in another embodiment, the present invention relates to a pharmaceutical composition comprising the compounds and/or nucleic acids and/or vectors of the invention and optionally a pharmaceutically acceptable carrier and/or diluent.

Examples of suitable pharmaceutical carriers are well known in the art (see for example U.S. Pat. No. 5,780,431 which specifically discloses pharmaceutical compositions comprising stabilized CRF preparations and which is therefore incorporated herein by reference) and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The compositions of the invention may be administered locally or systemically. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition.

Drugs or pro-drugs after their in vivo administration are metabolized in order to be eliminated either by excretion or by metabolism to one or more active or inactive metabolites (Meyer, J. Pharmacokinet. Biopharm. 24 (1996), 449-459). Thus, rather than using the actual compound or drug identified and obtained in accordance with the methods of the present invention a corresponding formulation as a pro-drug can be used which is converted into its active in the patient. Precautionary measures that may be taken for the application of pro-drugs and drugs are described in the literature; see, for review, Ozama, J. Toxicol. Sci. 21 (1996), 323-329.

As indicated above, experiments on anxiety- and depression-like behaviors using a compound of the present invention were performed. These experiments are described in detail in the examples, particularly in the behavior experiments in example 4. Surprisingly, we found that both cortagine and oCRF significantly decreased the immobility time when applied immediately before the preswim or before the testswim session. Opposite effects were expected on the basis of the reported findings that antagonism to CRFR1 decreases depression-like behavior in rodents as determined by FST (32), (33), as well as in humans (34). At the first glance, our results could be interpreted to mean that the employed CRFR1 agonists produced locomotor-stimulating effects rather than exhibited antidepressive effects. However, it is known that blockade of CRFR1 by nonpeptide antagonists such as CP-154,526 causes a nonspecific increase in locomotion in the EPM test (35). Similarly, hyperlocomotion was observed in CRFR1-deficient mice exposed to the open field (12). Since these finding clearly demonstrated that the elimination of CRFR1 causes a non-specific increase in explorative activity, it was expected that the selective stimulation of CRFR1 by cortagine would lead to decreased locomotor behavior. However, our study demonstrated that changes in locomotor behavior strongly depended on the paradigm employed. In particular, injection of cortagine into the lateral ventricles of the brain produced a decrease in locomotor activity on the EPM, whereas the same treatment in the FST resulted in apparently opposite effects. Moreover, administration of cortagine but not oCRF prior to the preswim session resulted in a decrease of the immobility lime during the testswim session 24 hours later. Since no correlation was found between immobility time during the pre- and testswim session, it is suggested that the cortagine-induced CRFR1 activity may in fact lead to a long-lasting decrease in depression-like behavior independently of its possible effects on locomotion. This finding is in sharp contrast to the prevailing hypothesis that increased CRFR1 activity results in increased susceptibility for the development of depression-like behavior in the FST (33).

Accordingly, the present invention also relates to the use of the compound and/or the nucleic acid and/or the vector of the invention for the preparation of a pharmaceutical composition for the treatment of (clinical) depression.

Depression is a disorder of mood, characterized by sadness and loss of interest in usually satisfying activities, a negative view of the self and hopelessness, passivity, indecisiveness, suicidal intentions, loss of appetite, weight loss, sleep disturbances, and other physical symptoms. Some or all of these symptoms may be present in people suffering from depression. Depression in many ways resembles the grief and mourning that follow bereavement, there are often feelings of low self esteem, guilt and self reproach, withdrawal from interpersonal contact and somatic symptoms such as eating and sleep disturbances. The term “clinical depression” refers to any form of depression that requires some form of treatment in order to alleviate it. The above mentioned meaning and scope of the terms depression or clinical depression is well-known to the skilled artisan (e.g. a physician or psychologist).

In a preferred embodiment, said depression is exogenic (like pharmacogenic), endogenic (like vital), psychogenic, agitated, anaclitic, arteriosclerotic, manic, reactive and/or senile depression.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease. The present invention is directed towards treating patients with medical conditions relating to depression. Accordingly, a treatment of the invention would involve preventing, inhibiting or relieving any medical condition related to depression.

The methods are applicable to both human therapy and veterinary applications. The compounds described herein having the desired therapeutic activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-100 wt %.

The administration of the pharmaceutical composition can be done in a variety of ways as discussed above, including, but not limited to, orally, subcutaneously, intravenously, intra-arterial, intranodal, intramedullary, intrathecal, intraventricular, intranasally, intrabronchial, transdermally, intranodally, intrarectally, intraperitoneally, intramuscularly, intrapulmonary, vaginally, rectally, or intraocularly. In some instances, for example, in the treatment of wounds and inflammation, the candidate agents may be directly applied as a solution dry spray.

Furthermore, it is envisaged by the present invention that the various nucleic acid molecules and vectors encoding the above described compounds (e.g. peptide-compounds) of the present invention are administered either alone or in any combination using standard vectors and/or gene delivery systems, and optionally together with a pharmaceutically acceptable carrier or excipient. For example, the nucleic acid molecule of the invention can be used alone or as part of a vector to express the compound(s) of the invention in cells, for, e.g., gene therapy. The nucleic acid molecules or vectors of the invention are introduced into the cells which in turn produce the compound. Subsequent to administration, said nucleic acid molecules or vectors may be stably integrated into the genome of the subject. On the other hand, viral vectors may be used which are specific for certain cells or tissues and persist in said cells. Suitable pharmaceutical carriers and excipients are well known in the art.

In the above-described embodiments, the vector of the present invention may preferably be a gene transfer or targeting vector. Gene therapy, which is based on introducing therapeutic genes, for example for vaccination into cells by ex-vivo or in-vivo techniques is one of the most important applications of gene transfer. Suitable vectors, methods or gene-delivering systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813, Isner, Lancet 348 (1996), 370-374; Muhihauser, Circ. Res. 77 (1995), 1077-1086; Onodua, Blood 91 (1998), 30-36; Verzeletti, Hum. Gene Ther. 9 (1998), 2243-2251; Verma, Nature 389 (1997), 239-242; Anderson, Nature 392 (Supp. 1998), 25-30; Wang, Gene Therapy 4 (1997), 393-400; Wang, Nature Medicine 2 (1996), 714-716; WO 94/29469; WO 97/00957; U.S. Pat. No. 5,580,859; U.S. Pat. No. 5,589,466; U.S. Pat. No. 4,394,448 or Schaper, Current Opinion in Biotechnology 7 (1996), 635-640, and references cited therein.

The nucleic acid molecules and vectors of the invention may be designed for direct introduction or for introduction via liposomes, or viral vectors (e.g. adenoviral, retroviral) into the cell. Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the nucleic acid molecules or vector of the invention into targeted cell populations. Preferably, said cell is a germ line cell, embryonic cell, or egg cell or derived therefrom, most preferably said cell is a stem cell. As mentioned above, suitable gene delivery systems may include liposomes, receptor-mediated delivery systems, naked DNA, and viral vectors such as herpes viruses, retroviruses, adenoviruses, and adeno-associated viruses, among others. Delivery of nucleic acids to a specific site in the body for gene therapy may also be accomplished using a biolistic delivery system, such as that described by Williams (Proc. Natl. Acad. Sci. USA 88 (1991), 2726-2729).

It is to be understood that the introduced nucleic acid molecules and vectors express the gene product after introduction into said cell and preferably remain in this status during the lifetime of said cell. For example, cell lines which stably express the nucleic acid molecule under the control of appropriate regulatory sequences may be engineered according to methods well known to those skilled in the art. Rather than using expression vectors which contain viral origins of replication, host cells can be transformed with the polynucleotide of the invention and a selectable marker, either on the same or separate plasmids. Following the introduction of foreign DNA, engineered cells may be allowed to grow for 1-2 days in an enriched media, and then are switched to a selective media. The selectable marker in the recombinant plasmid confers resistance to the selection and allows for the selection of cells having stably integrated the plasmid into their chromosomes and grow to form foci which in turn can be cloned and expanded into cell lines. A number of selection systems may be used, including but not limited to the herpes simplex virus thymidine kinase (Wigler, Cell 11(1977), 223), hypoxanthine-guanine phosphoribosyltransferase (Szybalska, Proc. Natl. Acad. Sci. USA 48 (1962), 2026), and adenine phosphoribosyltransferase (Lowy, Cell 22 (1980), 817) in tk⁻, hgprt⁻ or aprt⁻ cells, respectively. Also, antimetabolite resistance can be used as the basis of selection for dhfr, which confers resistance to methotrexate (Wigler, Proc. Natl. Acad. Sci. USA 77 (1980), 3567; O'Hare, Proc. Natl. Acad. Sci. USA 78 (1981), 1527), gpt, which confers resistance to mycophenolic acid (Mulligan, Proc. Natl. Acad. Sci. USA 78 (1981), 2072); neo, which confers resistance to the aminoglycoside G-418 (Colberre-Garapin, J. Mol. Biol. 150 (1981), 1); hygro, which confers resistance to hygromycin (Santerre, Gene 30 (1984), 147); or puromycin (pat, puromycin N-acetyl transferase). Additional selectable genes have been described, for example, trpB, which allows cells to utilize indole in place of tryptophan; hisD, which allows cells to utilize histinol in place of histidine (Hartman, Proc. Natl. Acad. Sci. USA 85 (1988), 8047); and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.).

In another embodiment, the present invention relates to a kit, preferably a diagnostic kit, comprising the compounds of the present invention. In addition, it is also envisaged that the compounds of the invention are comprised in a diagnostic composition. The components of the diagnostic composition and/or the kit of the invention may be packaged in containers such as vials, optionally in buffers and/or solutions. If appropriate, one or more of said components may be packaged in one and the same container. The parts of the kit of the invention can also be packaged individually in vials or in combination in containers or multicontainer units. Additionally or alternatively, one or more of said components may be adsorbed to solid support such as, e.g., a nitrocellulose filter or nylon membrane, or to the well of a microtiter plate. Solid phases are known to those in the art and may comprise polystyrene beads, latex beads, magnetic beads, colloid metal particles, glass and/or silicon chips and surfaces, nitrocellulose strips, membranes, sheets, animal red blood cells, or red blood cell ghosts, duracytes and the walls of wells of a reaction tray, plastic tubes or other test tubes. Suitable methods of immobilizing nucleic acids, (poly)peptides, proteins, antibodies, etc. on solid phases include but are not limited to ionic, hydrophobic, covalent interactions and the like.

The kit of the invention may advantageously be employed in a variety of applications referred to herein, e.g., in the diagnostic field or as research tool. Manufacture of the kit follows preferably stahdard procedures which are known to the person skilled in the art. In another embodiment it is envisaged that the kit of the present invention comprises instructions to use.

The present invention also relates to the use of the compound of the present invention for the preparation of a diagnostic composition for the determination of pituitary corticotroph responsiveness.

In another embodiment, the present invention relates to the use of the compounds of the invention for the preparation of a diagnostic composition for differentiating pituitary and ectopic production of ACTH in patients with ACTH-dependent Cushing's syndrome.

It is well-known that there are two forms of Cushing's syndrome:
(a) ACTH-dependent, in which hypercortisolism is due either to pituitary hypersecretion of ACTH (Cushing's disease) resulting from an adenoma (40%, usually microadenomas) or nonadenomatous hyperplasia, possibly of hypothalamic origin (28%), or to hypercortisolism that is secondary to ectopic secretion of ACTH (15%) and,
(b) ACTH-independent (17%), in which hypercortisolism is due to autonomous cortisol secretion by an adrenal tumor (9% adenomas, 8% carcinomas).

After the establishment of hypercortisolism consistent with the presence of Cushing's syndrome, and following the elimination of autonomous adrenal hyperfunction as its cause, the corticorelin test is used to aid in establishing the source of excessive ACTH secretion.

This disclosure may best be understood in conjunction with the accompanying drawings, incorporated herein by references. Furthermore, a better understanding of the present invention and of its many advantages will be had from the following examples, given by way of illustration and are not intended as limiting.

Abbreviations

Abbreviations used throughout the description, the figures, the figure legends, the tables, the table legends, the claims and the examples are as follows:
corticotropin-releasing factor, CRF
human/rat CRF, h/rCRF
ovine CRF, OCRF
sauvagine, Svg
urocortin, Ucn
mouse Ucn, mucn
CRF receptor, CRFR
rat CRFR, rCRFR
mouse CRFR, mCRFR
CRF binding protein, CRFBP
rat CRFBP, rCRFBP
anti-sauvagine-30, aSvg-30
intracerebroventricular, i.c.v.
intraseptal, i.s.
artificial cerebrospinal fluid, aCSF
elevated plus-maze, EPM
forced swim test, FST

IUPAC rules are used for the nomenclature of peptide-compounds including one and three-letter codes for amino acids.

The figures show:

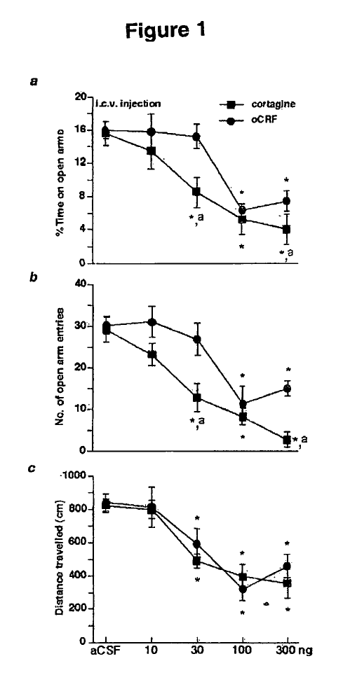

FIG. 1: Enhancement of anxiety-like behavior by cortagine
(Potency of the selective CRFR1 agonist cortagine is higher compared to oCRF in the plus-maze behavior of C57BL/6J mice)
I.c.v. administration of 300 ng (68 pmol), 100 ng (23 pmol) and 30 ng (6.8 pmol) cortagine produced increased anxiety levels and reduced locomotor activity as indicated by the time spent on the open arms (a), number of entries into the open arms (b) and total distance travelled (cm) (c) in the EPM. I.c.v. administration of 300 ng (68 pmol) and 100 ng (21 pmol) oCRF also significantly decreased the time spent on the open arms (a), number of entries into the open arms (b) and total distance travelled (cm) (c) in the EPM. The doses of 30 ng (6.4 pmol) and 10 ng (2.1 pmol) oCRF proved to be ineffective whereas a dose as low as 30 ng cortagine was anxiogenic. The agonists were injected i.c.v. 30 min before exposure to the EPM for 5 min. Statistically significant differences: Bonferroni/Dunn test, *p<0.05 relative to control (aCSF); ₐp<0.05 relative to oCRF at respective dose.

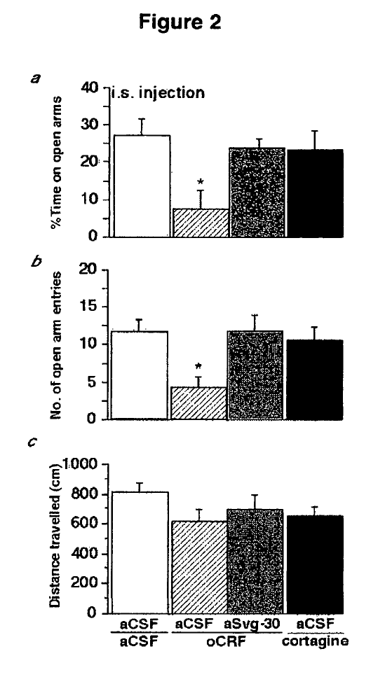

FIG. 2: Absence of significant interaction of cortagine with CRFR2 of the mouse brain
(Cross-reactivity to CRFR2 of the selective CRFR1 agonist cortagine is lower compared to oCRF in the plus-maze behavior of C57BL/6J mice)
I.s. administration of 100 ng (21 pmol) oCRF, but not cortagine, produced increased anxiety levels as indicated by the time spent on the open arms (a), number of entries into the open arms (b), without affecting locomotor activity (c) in the EPM. Anxiogenic effects produced by 100 ng (21 pmol) oCRF were fully antagonized by 400 ng (110 pmol) aSvg-30 (a, b). The antagonist or a CSF were injected i.s. 45 and 30 min, respectively, before exposure to the EPM for 5 min. Statistically significant differences: Bonferroni/Dunn test, *p<0.05 vs control (aCSF).

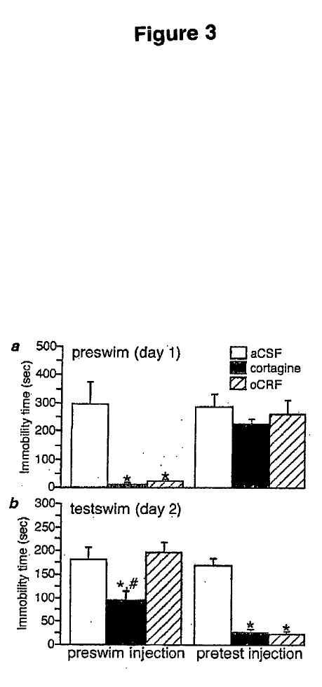

FIG. 3: Anti-depression-like activity of cortagine in the Forced Swim Test
I.c.v. administration of 300 ng (68 pmol) cortagine or 300 ng (64 pmol) oCRF 30 min before the preswim on day 1 significantly decreased the immobility time during the preswim compared with aCSF administration (a). Only mice treated with cortagine on day 1 displayed significant decrease of immobility time during the testswim 24 h later (day 2) (b).
I.c.v. administration of 300 ng (68 pmol) cortagine or 300 ng (64 pmol) oCRF 30 min before the testswim on day 2 significantly decreased immobility time during the testswim (b). Statistically significant differences: Bonferroni/Dunn test, *p<0.05 vs control (aCSF); ^{#}p<0.05 vs oCRF-injected group.

The tables show:

FIG. 4. Sequence alignment of oCRF, h/rCRF, Svg and their chimeric analogs

FIG. 5. Binding affinites of oCRF, h/rCRF, Svg and their chimeric analogs

FIG. 6. Comparison of the pharmacological and physicochemical properties of cortagine and oCRF

### EXAMPLES

The following examples illustrate the invention. These examples should not be construed as to limit the scope of this invention. The examples are included for purposes of illustration and the present invention is limited only by the claims.

### Example 1

### Peptide Synthesis

Peptides were synthesized, purified, and characterized by mass spectrometry as described recently (20, 36).

### Example 2

### Biological Potency of Coltagine

The biological potency of cortagine and OCRF was evaluated by the determination of the EC₅₀ values for agonist-induced intracellular accumulation of cAMP in HEK 293 cells producing either rCRFR1 or mCRFR2β. In agreement with the binding data, the biological potencies of cortagine and oCRF were high at rCRFR1 and about one to two orders of magnitude lower at mCRFR2β (FIG. 6).

A) Binding Assays

Crude membrane fractions were prepared from human embryonic kidney (HEK) 293 cells stably expressing either cloned rat CRFR1α(rCRFR1) or mouse CRFR2β (mCRFR2β) as described (20). Rat CRFBP (rCRFBP) was produced in HEK 293 cells stably transfected with cDNA coding for rCRFBP C-terminally fused with a His₆ sequence as described (36). The scintillation proximity assays (SPA) for binding analysis of CRFR and CRFBP have been already described in detail (21, 22). For competition binding assays of rCRFR1 and rCRFBP, [¹²⁵I-Tyr⁰]h/rCRF was used as radiolabeled peptide, whereas [¹²⁵I-Tyr⁰]Svg was employed for mCRFR2β.

B) Measurement of Intracellular cAMP Accumulation

The HEK 293 cells were plated into 24 well cell culture plates and stimulated as described (21) using increasing concentrations of the agonists under investigation. Intracellular cAMP was measured with the Biotrak™ cAMP [¹²⁵I] SPA system (Amersham Pharmacia Biotech) according to the manufacturer's product manual.

### Example 3

### Determination of Maximum Solubility and Isoelectric Point

After dissolving the peptides in artificial cerebrospinal fluid (aCSF; 124 mM NaCl, 26.4 mM NaHCO₃, 10 mM glucose, 3.3 mM KCl, 2.5 mM CaCl₂ , 2.4 mM MgSO₄ , 1.2 mM KH₂PO₄) at pH=7.4, the maximum solubility cₘₐₓ was determined by amino acid analysis as described (21). The isoelectric point of the peptides was determined by isoelectric focusing (IEF) as described (21) with a Bio-Rad IEF cell system using Bio-Rad IEF strips in the pH range 3-10.

Solubility of Cortagine

A high solubility under physiological conditions is a crucial prerequisite especially for compounds to be injected into the rodent brain. Therefore, the maximal solubility cₘₐₓ of cortagine and oCRF in aCSF was determined. Both peptides were soluble in a concentration range of up to 1000 μM (FIG. 6), so that there was no limitation in view of the agonist doses typically used in behavioral experiments. Interestingly, cortagine, but not oCRF, was found to be insoluble when stock solutions in 10 mM aqueous acetic acid were prepared for the pharmacological assays. The low solubility of cortagine under acidic conditions was attributed to its relatively acidic isoelectric point (pl=4.8; FIG. 6) that was determined by IEF. Consequently, solutions of cortagine to be used in the pharmacological assays were prepared in phosphate buffered saline (PBS; pH=7.4). Although soluble in 10 mM aqueous acetic acid, oCRF was also dissolved in PBS to meet the same experimental conditions as for cortagine.

### Example 4

### Behavioral Experiments

A) Animals

Nine-week old male C57BL/6J (Centre D'Elevage Janvier, Sultzfeld, France) were individually housed in macrolon cages according to the recommendations of the Society for Laboratory Animal Science (Germany). All experiments were carried out in accordance with the European Council Directive (86/609/EEC) with the permission of the Animal Protection Law enforced by the District Government of Braunschweig, State of Lower Saxony, Germany, which is in full agreement with the APA ethical guidelines. All efforts were made to minimize animal suffering. The number of mice per group was 9-11.

B) Preparation of Peptide Solutions for Behavioral Experiments

All peptides were dissolved in aCSF except for the CRFR2-selective antagonist antisauvagine-30 (aSvg-30) (20) that was initially dissolved in 10 mM aqueous acetic acid and diluted with the same volume of two-fold concentrated aCSF. The final pH of the peptide solutions was 7.4. The exact peptide concentration of the injection solutions was determined by amino acid analysis with a Beckman HPLC analyzer system 6300. Total hydrolysis of the peptides was carried out in the presence of 1 nmol norvaline as internal standard with 6 M HCl for 24 h at 100° C.

C) The Elevated Plus-maze Test

The elevated plus-maze (EPM) behavior of C57BL/6J mice cannulated in the lateral ventricles or lateral intermediate septal area (23) was investigated 30 min after injection of the CRFR1 agonist under investigation for 5 min in the EPM test (24). The CRFR2-selective antagonist aSvg-30 in aCSF or aCSF alone were injected 15 min before agonist administration. The behavior of the mice was recorded by a video camera connected to a PC and analyzed by the Technical & Scientific Equipment software VIDEOMOT 2. The time spent, distance crossed, and number of entries in the open arms, closed arms, and center were recorded. The cannular placement was confirmed for each mouse by histological examination of the brains after methylene blue injection (23). The behavioral data are expressed as mean±SEM, and were analyzed using a two- and one-way ANOVA, with Bonferroni/Dunn test applied, post hoc, for individual between-group comparisons at the p<0.05 level of significance. Shift of preference from the open to the closed arms was interpreted as an increase of anxiety-like behavior. Locomotor activity was determined with this test by the distance travelled.

D) The Forced Swim Test

For the forced swim test (FST), C57BL/6J mice cannulated in the lateral ventricles were subjected to swim sessions in individual glass cylinders (height 39 cm, diameter 21.7 cm) containing water, 15-cm deep, at 23-25° C. On day 1, all animals were placed in the cylinder for a preswim session of 15 min. On the test day 24 h later (day 2), the mice were subjected to a testswim session for 6 min. The water was changed between subjects. All testswim sessions were recorded by a video camera positioned directly above the cylinder. A component observer blind to treatment scored the videotapes. The behavioral measure scored was the duration of immobility, defined as time spent still or only using righting movements to keep the head over water. An increase in immobility time was interpreted as an increase of depression-like behavior. In all behavioral experiments, the cannula placement was confirmed for each mouse by histological examination of the brains after methylene blue injection (23). The behavioral data are expressed as mean±SEM, and were analysed using a two- and one-way ANOVA, with Bonferri/Dunn test applied, post hoc, for individual between-group comparisons at the p<0.05 level of significance.

Behavioral Effects of Cortagine

On the basis of the pharmacological data presented, an in vivo potency higher than that of oCRF could be expected for cortagine in view of behavioral effects mediated by CRFR1. Since the significance of CRFR1 in anxiety (11, 12) and locomotion (28) is established, we addressed this question in a series of behavior experiments by using the EPM test and the FST.

a) Modulation of Anxiety-like Behavior by Cortagine

A two-way ANOVA using treatment and dose as between-subject factors indicated significant treatment, dose, and interaction effects, after administration of peptides into the lateral ventricles (i.c.v.) of male C57BL/6J mice 30 min before testing, on time spent in open arms (F_{(1, 80)}=6.55; p<0.05 for treatment; F_{(4, 80)}=48.29; p<0.05 for dose; F_{(4, 80)}=3.71; p<0.05 for interaction) (FIG. 1a) and number of open arms entries (F_{(1, 80)}=4.69; p<0.05 for treatment; F_{(4, 80)}=20.37; p<0.05 for dose; F_{(4, 80)}=4.38; p<0.05 for interaction) (FIG. 1b) of the EPM. These results revealed a significantly higher anxiogenic potency of cortagine compared to oCRF (Bonferroni/Dunn test, p<0.05, for time spent and number of open arm entries of the EPM). An interaction effect was due to differences between 30 ng cortagine and oCRF in modulating anxiety-like behavior in the EPM as confirmed by analyses of simple effects of dose. In particular, 30 ng cortagine but not oCRF significantly decreased time spent in open arms (F_{(1, 18)}=15.37; p<0.05) (Bonferroni/Dunn test, p<0.05) and number of open arms entries (F_{(1, 18)}=10.89; p<0.05) (Bonferroni/Dunn test, p<0.05) of the EPM. Interestingly, the peptides tested did not differ in their ability (F_{(1, 80)}=0.85; p>0.05) to modulate locomotor activity (FIG. 1c).

Analysis of simple effects of treatment showed that i.c.v. administration of 300 ng (67 pmol), 100 ng (22 pmol), and 30 ng (6.6 pmol) cortagine increased significantly anxiety-like behavior as indicated by a decreased time spent in open arms (F_{(4, 40)}=19.48; p<0.05) (Bonferroni/Dunn test, p<0.05 vs aCSF) (FIG. 2a) and number of open arms entries (F_{(4,40)}=7.84; p<0.05; Bonferroni/Dunn test, p<0.05 vs aCSF) (FIG. 2b) of the EPM. At those three doses locomotor activity was also decreased significantly as revealed by the total distance crossed (F_{(4,40)}=16.02; p<0.05) (Bonferroni/Dunn test, p<0.05 vs aCSF) (FIG. 2c). However, 10 ng (2.2 pmol) cortagine were ineffective, pointing to a dose-dependent response to the peptide. I.c.v. administration of 300 ng (67 pmol) and 100 ng (22 pmol) oCRF produced a similar anxiogenic effect, as indicated by a decreased time spent (F_{(4,40)}=36.55; p<0.05) (Bonferroni/Dunn test, p<0.05 vs aCSF) and number of entries into the open arms (F_{(4,40)}=17.31; p<0.05) (Bonferroni/Dunn test, p<0.05 vs aCSF) (FIG. 1a, b) in the EPM, and affected maze locomotor activity as revealed by the total distance crossed (F_{(4,40)}=9.46; p<0.05) (Bonferroni/Dunn test, p<0.05 vs aCSF) (FIG. 1c). Interestingly, 30 ng (6.6 pmol) and 10 ng (2.2 pmol) oCRF did not produce any significant behavioral effect in the EPM, indicating that the minimal doses required for changes in anxiety differed between the two peptides.

We also investigated whether a possible cross-reactivity of cortagine to CRFR2 is less significant than that of oCRF by monitoring the plus-maze behavior of C57BL/6J mice. Administration of 100 ng (22 pmol) oCRF, but not of 100 ng (22 pmol) cortagine into the lateral septum (i.s.), containing a high density of CRFR2α but lacking CRFR1 (30), 30 min before testing in the EPM, exerted a profound anxiogenic effects as indicated by a decreased time spent in open arms (F_{(4,42)}=7.32; p<0.05) (Bonferroni/Dunn test, p<0.05 vs aCSF) and number of open arm entries (F_{(4,42)}=6.34; p<0.05) (Bonferroni/Dunn test, p<0.05 vs aCSF) (FIG. 2a, b), without affecting locomotor activity (F_{(4,42)}=1.16; p>0.05) (FIG. 2c) in the EPM. Thus, the differences between the peptides in their ability to activate CRFR2 observed in vitro (FIG. 6) were confirmed by the behavioral observations in vivo. When 400 ng of the CRFR2-selective antagonist aSvg-30 were injected i.s. 15 min before the application of 100 ng oCRF, effects of the peptide in the EPM were completely prevented. In agreement with an earlier study (23), it was concluded on the basis of the specificity of aSvg-30 that anxiogenic effects of oCRF were probably mediated by septal CRFR2.

b) Modulation of the Depression-like Behavior in the Forced Swim Test

Previous studies have demonstrated that antagonism of CRFR1 decreases the immobility time in the FST, a rodent model of depression-like behavior (32), (33). To examine the effect of selective activation of CRFR1 on depression-like behavior, C57BL/6J mice were injected with cortagine and tested in the FST. One group of mice was injected i.c.v. with 300 ng (68 pmol) cortagine or 300 ng (64 pmol) oCRF 30 min before the preswim session (day 1) and examined in the testswim session 24 h later (day 2). The second group of mice was exposed to the preswim session without injection (day 1). However, 300 ng (68 pmol) cortagine or 300 ng (64 pmol) oCRF were administered 30 min prior to the testswim session 24 h later (day 2). Interestingly, a two-way ANOVA with treatment and order (pre-swim vs pretestswim injection) as between-subject factors revealed significant treatment and order main effects and treatment×order interaction for immobility time during the preswim session (F_{(2,41)}=10.24; p<0.05 treatment; F_{(1,41)}=19.52; p<0.05 order; F_{(2,41)}=5.63; p<0.05 treatment×order) and testswim session (F_{(2,41)}=19.93; p<0.05 treatment; F_{(1,41)}=34.07; p<0.05 order; F_{(1,41)}=10.65; p<0.05 treatment×order) in the FST. Bonferroni/Dunn post hoc analysis showed that pre-preswim and/or pre-testswim treatment with cortagine or oCRF significantly decreased the immobility time compared with aCSF-injected mice (p<0.05 vs aCSF) (FIG. 4a, b). Similarly, pre-preswim injection of the peptides significantly decreased the immobility time during the preswim session (p<0.05 vs testswim session), whereas the pre-testswim injection exerted the corresponding effect during the testswim session (p<0.05 vs preswim session) (FIG. 4a, b). Initially, these results suggested that selective activation of CRFR1 was not essential for depression-like behavior but may contribute to the pharmacological activation of CRF pathways relevant to non-specific increase in locomotion. However, analysis of simple main effects of treatment revealed that cortagine injection prior to the preswim session resulted in a significantly reduced immobility time in comparison to oCRF-injected mice during the testswim session 24 h later (F(2,22)=5.94; p<0.05) (Bonferroni/Dunn test, #p<0.05 vs oCRF-injected group) (FIG. 4b). No such difference was observed between cortagine and oCRF-pretreated mice during the preswim session (F(2,22)=9.65; p<0.05) (Bonferroni/Dunn test, p>0.05 cortagine- vs oCRF-injected group) (FIG. 4a). This result indicated that in contrast to oCRF, cortagine may produce long lasting effects on depression-like behavior. Since no significant correlation between immobility time during pre- and testswim session was found (r=−0.45, p>0.05), the observed cortagine-mediated action on depression-like behavior may be independent from its apparent effect on locomotor activity.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference.

### REFERENCES

1. Spiess, J., Rivier, J., Rivier, C. & Vale, W. W. (1981) Proc. Natl. Acad. Sci. USA 78, 6517-6521.
2. Vale, W. W., Spiess, J., Rivier, C. & Rivier, J. (1981) Science 213, 1394-1397.
3. Eckart, K., Jahn, O., Radulovic, J., Radulovic, M., Blank, T., Stiedl, O., Brauns, O., Tezval, H., Zeyda, T. & Spiess, J. (2002) Receptors Channels 8, 163-177. 24
4. Reul, J. M. & Holsboer, F. (2002) Curr. Opin. Pharmacol. 2, 23-33
5. Perrin, M. H. & Vale, W. W. (1999) Annals of the New York Academy of Sciences 885, 312-328.
6. Behan, D. P., Heinrichs, S. C., Troncoso, J. C., Liu, X. J., Kawas, C. H., Ling, N. & De Souza, E. B. (1995) Nature 378, 284-287.
7. Karolyi, I. J., Burrows, H. L., Ramesh, T. M., Nakajima, M., Lesh, J. S., Seong, E., Camper, S. A. & Seasholtz, A. F. (1999) Proc. Natl. Acad. Sci. USA 96, 11595-11600.
8. Hillhouse, E. W., Randeva, H., Ladds, G. & Grammatopoulos, D. (2002) Biochem. Soc. Trans. 30, 428-432.
9. Lovenberg, T. W., Chalmers, D. T., Liu, C. & De Souza, E. B. (1995) Endocrinology 136, 4139-4142.
10. Bale, T. L., Contarino, A., Smith, G. W., Chan, R., Gold, L. H., Sawchenko, P.

E., Koob, G. F., Vale, W. W. & Lee, K. F. (2000) Nat. Genet. 24, 410-414.
11. Smith, G. W., Aubry, J. M., Dellu, F., Contarino, A., Bilezikjian, L. M., Gold, L. H., Chen, R., Marchuk, Y., Hauser, C., Bentley, C. A., Sawchenko, P. E., Koob, G. F., Vale, W. W. & Lee, K. F. (1998) Neuron 20, 1093-1102.
12. Timpl, P., Spanagel, R., Sillaber, I., Kresse, A., Reul, J., Stalla, G. K., Blanquet, V., Steckler, T., Holsboer, F. & Wurst, W. (1998) Nat. Genet. 19, 162-166.
13. Kishimoto, T., Radulovic, J., Radulovic, M., Lin, C. R., Schrick, C., Hooshmand, F., Hermanson, O., Rosenfeld, M. G. & Spiess, J. (2000) Nat. Genet. 24, 415-419.
14. Koob, G. F. (1999) Biol. Psychiatry 46, 1167-80)
15. Vaughan, J., Donaldson, C., Bittencourt, J., Perrin, M. H., Lewis, K., Sutton, S., Chan, R., Turnbull, A. V., Lovejoy, D., Rivier, C., Rivier, J., Sawchenko, P. E. & Vale, W. W. (1995) Nature 378, 287-292.
16. Behan, D. P., Grigoriadis, D. E., Lovenberg, T., Chalmers, D., Heinrichs, S., Liaw, C. & De Souza, E. B. (1996) Mol. Psychiatry 1, 265-277.
17. Reyes, T. M., Lewis, K., Perrin, M. H., Kunitake, K. S., Vaughan, J., Arias, C. A., Hogenesch, J. B., Gulyas, J., Rivier, J., Vale, W. W. & Sawchenko, P. E. (2001) Proc. Natl. Acad. Sci. USA 98, 2843-2848.25
18. Hsu, S. Y. & Hsueh, A. J. W. (2001) Nat Med 7, 605-611.
19. Lewis, K., Li, C., Perrin, M. H., Blount, A., Kunitake, K., Donaldson, C., Vaughan, J., Reyes, T. M., Gulyas, J., Fischer, W., Bilezikjian, L., Rivier, J., Sawchenko, P. E. & Vale, W. W. (2001) Proc. Natl. Acad. Sci. USA 98, 7570-7575.
20. Rühmann, A., Bonk, I., Lin, C. J. R., Rosenfeld, M. G. & Spiess, J. (1998) Proc. Natl. Acad. Sci. USA 95, 15264-15269.
21. Eckart, K., Jahn, O., Radulovic, J., Tezval, H., van Werven, L. & Spiess, J. (2001) Proc. Natl. Acad. Sci. USA 98 11142-11147
22. Hofmann, B. A., Sydow, S., Jahn, O., van Werven, L., Liepold, T., Eckart, K. & Spiess, J. (2001) Prot. Sci. 10, 2050-2062.
23. Radulovic, J., Rühmann, A., Liepold, T. & Spiess, J. (1999) J Neurosci. 19, 5016-5025.
24. Radulovic, J., Kammermeier, J. & Spiess, J. (1998) Behav. Brain Res. 95, 179-189.
25. Beyermann, M., Rothemund, S., Heinrich, N., Fechner, K., Furkert, J., Dathe, M., Winter, R., Krause, E. & Bienert, M. (2000) J. Biol. Chem. 275, 5702-5709.
26. Brauns, O., Brauns, S., Zimmermann, B., Jahn, O. & Spiess, J. (2002) Peptides 23, 881-888.
27. Saido, T. C., Yamaoharigaya, W., Iwatsubo, T. & Kawashima, S. (1996) Neurosci Lett 215, 173-176.
28. Contarino, A., Dellu, F., Koob, G., Smith, G., Lee, K., Vale, W. & Gold, L. (2000) Endocrinology 141, 2698-2702.
29. Chalmers, D. T., Lovenberg, T. W. & De Souza, E. B. (1995) J. Neurosci. 15, 6340-6350.
30. Dunn, A. J. & Berridge, C. W. (1990) Brain Res. Brain Res. Rev. 15, 71-100.
31. Van Pett, K., Viau, V., Bittencourt, J. C., Chan, R. K. W., Li, H. Y., Arias, C., Prins, G. S., Perrin, M., Vale, W. & Sawchenko, P. E. (2000) J. Comp. Neurol. 428, 191-212.
32. Griebel, G., Simiand, J., Steinberg, R., Jung, M., Gully, D., Roger, P., Geslin, M., Scatton, B., Maffrand, J. P. & Soubrie, P. (2002) J. Pharmacol. Exp. Ther. 301, 333-345.
33. Bale, T. L. & Vale, W. W. (2003) J. Neurosci. 23, 5295-5301
34. Zobel, A. W., Nickel, T., Kunzel, H. E., Ackl, N., Sonntag, Al, Ising, M. & Holsboer, F. (2000), J. Psychiatr. Res. 34, 171-81
35. Griebel, G., Perrault, G. & Sanger, D. J. (1998) Psychopharmacology 138, 55-66.
36. Jahn, O., Eckart, K., Sydow, S., Hofmann, B. A. & Spiess, J. (2001) Peptides 22, 47-56.

1. A compound which is highly selective for CRFR1 without having any significant cross-reactivity for corticotropin-releasing-factor-receptor-2 (CRFR2) and/or corticotropin-releasing-factor-binding protein (CRFBP), said compound comprising or alternatively consisting of the amino acid sequence
Glx¹⁻Gly²⁻pro³⁻pro⁴⁻Xaa⁵⁻Ser⁶⁻Xaa⁷⁻Asp⁸⁻Leu⁹⁻Xaa¹⁰⁻Leu¹¹⁻Glu¹²⁻Leu¹³⁻Leu⁴⁻Arg¹⁵⁻Glu¹⁶⁻Val¹⁷⁻Leu¹⁸⁻Glu¹⁹⁻Xaa²⁰⁻Xaa²¹⁻Arg²²⁻Ala²³⁻Xaa²⁴⁻Gln²⁵⁻Leu²⁶⁻Ala²⁷⁻Gln²⁸⁻Gln²⁹⁻Ala³⁰⁻Ala¹¹⁻Asn³²⁻Asn³³⁻Arg³⁴⁻Leu³⁵⁻Leu³⁶⁻Leu³⁷⁻Asp³⁸⁻Thr³⁹⁻Ala⁴⁰(SEQID No: 1).

2. The compound of claim 1 wherein:
(a) Xaa⁵ is lie, Leu or any amino acid residue having similar physicochemical characteristics as lie; and/or
(b) Xaa⁷ islle, Leu or an amino acid residue having similarphysicochemical characteristics as Ile; and/or
(c) Xaa¹⁰ is Ser, Thr or an amino acid residue having similar physicochemical characteristics as Serin; and/or
(d) Xaa²⁰ is Met, Norleucine or any amino acid residue having similar physicochemical characteristics as Met; and/or
(e) Xaa²¹ is Glu, Asp or an amino acid residue having similarphysicochemical characteristics as Glu; and/or
(f) Xaa²⁴ is Glu, Asp or an amino acid residue having similarphysicochemical characteristics as Glu.

3. The compound of claim 1 or 2 which is Glx¹⁻Gly²⁻Pro³⁻Pro⁴⁻Ile⁵⁻Ser⁶⁻Ile⁷⁻Asp⁸⁻Leu⁹⁻Ser¹⁰⁻Leu¹¹⁻Glu¹²⁻Leu¹³⁻Leu¹⁴⁻Arg¹⁵⁻Glu¹⁶⁻Val¹⁷Leu¹⁸⁻Gluj ⁹⁻Met²⁰⁻Glu²¹⁻Arg²²⁻Ala²³⁻Glu²⁴⁻Gln²⁵⁻Leu²⁶⁻Ala²⁷⁻Gin²⁸⁻Gln²⁹⁻Ala³⁰⁻Ala³¹⁻Asn³²⁻Asn³³⁻Arg³⁴⁻Leu³⁵⁻Leu³⁶⁻Leu³⁷⁻Asp³⁸⁻Thr³⁹⁻Aia⁴⁰(SEQ ID No: 2).

4. A nucleic acid molecule encoding the compound of claim 1.

5. A vector comprising the nucleic acid molecule of claim 4.

6. The compound of claim 1 which is labelled.

7. The compound of claims 1 which is modified by:
(a) formation of pharmaceutical acceptable salts;
(b) formation ofpharmaceutically acceptable complexes; and/or
(c) synthesis ofpharnacologically active polymers.

8. A pharmaceutical composition comprising the compound of claim 1 and/or the nucleic acid and/or the vector and optionally a pharmaceutical acceptable carrier and/or diluent.

9. A diagnostic composition comprising the compound of claim 1.

10. A kit comprising the compound of claim 1 and/or the nucleic acid and/or the vector and optionally instructions to use.

11. Use of the compound of claim 1 and/or the nucleic acid and/or the vector for the preparation of a pharmaceutical composition for the treatment of depression.

12. The use of claim 11, wherein said depression is exogenic (like pharmacogenic), endogenic (like vital), psychogenic, agitated,anaclitic, arteriosclerotic, reactive and/or senile depression.

13. Use of the compound of claim 1 for the preparation of a diagnostic composition for the determination of pituitary corticotroph responsiveness.

14. The use of claim 13 for differentiating pituitary and ectopic production of ACTH in patients with ACTH-dependent Cushing's syndrome.
